(19) United States
(12) Patent Application Publication
(10) Pub. No.: US 2024/0132081 A1
(43) Pub. Date: Apr. 25, 2024

(54) EMOTIONAL STATE ASSESSMENT WITH DYNAMIC USE OF PHYSIOLOGICAL SENSOR RESPONSES

(71) Applicant: GM Global Technology Operations LLC, Detroit, MI (US)

(72) Inventors: Mia Levy, Los Angeles, CA (US); Evelyn Kim, Irvine, CA (US); Rajan Bhattacharyya, Sherman Oaks, CA (US)

(21) Appl. No.: 18/048,242

(22) Filed: Oct. 19, 2022

(51) Int. Cl. B60W 50/00; A61B 5/16; A61B 5/18; B60W 40/08; B60W 50/14; G06N 3/04

(52) U.S. Cl. CPC B60W 50/0098; A61B 5/163; A61B 5/18; B60W 40/08; B60W 50/0097; B60W 50/14; G06N 3/049; B60W 2040/0872; B60W 2050/0083; B60W 2050/146; B60W 2540/22; B60W 2540/221; B60W 2540/225

(57) ABSTRACT

A system for dynamically adjusting interactions between an ADAS equipped vehicle and occupants of the vehicle includes one or more physiological sensors disposed on the vehicle and one or more control modules having a processor, a memory, and input/output (I/O) ports in communication with the one or more physiological sensors. The control modules execute program code portions stored in the memory that: collect sensor data from the one or more physiological sensors; analyze the sensor data and select a subset of the sensor data corresponding to a subset of the one or more physiological sensors; predicts, based on the subset of the sensor data, that an occupant of the vehicle is experiencing an increase in stress level; and adapt an ADAS action of the vehicle to reduce an occupant stress level from a first level to a second level lower than the first level.

### INTRODUCTION

The present disclosure relates to vehicle sensors and communications systems, and more specifically to data collection systems and methods for vehicles with advanced driver assistance systems. Vehicles are increasingly being equipped with networked systems that communicate both internally, i.e. onboard, and externally, i.e. with other vehicles or remotely-located infrastructure via a variety of wireless and/or wired communication systems. Vehicle data collection systems generate or capture data relating to a wide range of vehicle attributes, from entertainment and climate-control functions to vehicle dynamics and safety systems. Data collection systems, both onboard and remote, receive large quantities of data relating to vehicle and atmospheric condition attributes. The data collection systems may then report relevant data to databases for further analysis, or may provide feedback to the vehicle control and communications systems based on prior information. More specifically, vehicles and/or infrastructure may collect information regarding vehicle user emotional states when using advanced driver assistance systems in vehicles. However, the data collection by such vehicles and infrastructure is often inhibited by monetary and computational cost constraints, signal quality, and reaction times.

While current advanced driver assistance systems achieve their intended purpose, there is a need for a new and improved system and method for assessing the emotional states of users operating vehicles with advanced driver assistance systems that improves vehicle user comfort and allows for platform and vehicle flexibility, upgradability on both the vehicular end and the remote end of the system, and which operate on preexisting hardware as well as new hardware while maintaining or decreasing the complexity of manufacture, assembly, and operation.

### SUMMARY

According to an aspect of the present disclosure a system for dynamically adjusting interactions between an advanced driver assistance system (ADAS) equipped vehicle and occupants of the vehicle includes one or more physiological sensors disposed on the vehicle. The system further includes one or more control modules having a processor, a memory, and input/output (I/O) ports, the I/O ports in communication with the one or more physiological sensors. The control modules execute program code portions stored in the memory. The program code portions include a first program code portion that collects sensor data from the one or more physiological sensors, and a second program code portion including a machine learning algorithm that analyzes the sensor data and selects a subset of the sensor data corresponding to a subset of the one or more physiological sensors. A third program code portion predicts, based on the subset of the sensor data, that an occupant of the vehicle is experiencing an increase in stress level. A fourth program code portion that adapts an ADAS action of the vehicle to reduce an occupant stress level from a first level to a second level lower than the first level.

In another aspect of the present disclosure the second program code portion further selects the subset of the sensor data corresponding to the subset of the one or more physiological sensors such that computational resource usage is maintained or decreased from a first computational resource usage level to a second computational resource usage level less than the first computational resource usage level.

In a yet another aspect of the present disclosure the second program code portion includes Shapley Additive Explanations (SHAP) to select the subset of sensor data corresponding to the subset of the one or more physiological sensors.

In a yet another aspect of the present disclosure the third program code portion maintains accuracy of stress level predictions while using subset of the sensor data from the subset of the one or more physiological sensors.

In a yet another aspect of the present disclosure the second program code portion includes long short-term memory (LSTM) to predict the stress level of the occupant of the vehicle.

In a yet another aspect of the present disclosure upon detecting an increase in occupant stress level in response to an ADAS action of the vehicle, the fourth program code portion actively, recursively, and continuously modulates the ADAS action to reduce the occupant stress level from the first level to the second level.

In a yet another aspect of the present disclosure the one or more physiological sensors comprise: electroencephalographs (EEGs), functional near-infrared spectroscopy (fNIRS) sensors, galvanic skin response (GSR) sensors, pupil diameter sensors, heart rate sensors, eye movement sensors, thermal camera, web camera, and photoplethysmography (PPG) sensors.

In a yet another aspect of the present disclosure the subset of the one or more physiological sensors includes the GSR sensor detecting changes in galvanic skin response, the pupil diameter sensor detecting changes in diameter of the occupant's pupil, and eye movement sensors detecting changes in eye position and rate of movement in X and Y directions.

In a yet another aspect of the present disclosure the control logic further includes a fifth control logic that actively informs the occupant, via a human-machine interface, of planned adaptations to the ADAS actions of the vehicle.

In a yet another aspect of the present disclosure a method for dynamically adjusting interactions between an advanced driver assistance system (ADAS) equipped vehicle and occupants of the vehicle includes collecting sensor data from one or more physiological sensors disposed on the vehicle. The method further includes utilizing one or more control modules having a processor, a memory, and input/output (I/O) ports, the I/O ports in communication with the one or more physiological sensors. The control modules executing program code portions stored in the memory. The program code portions analyze the sensor data and select a subset of the sensor data corresponding to a subset of the one or more physiological sensors, and predict, based on the subset of the sensor data, that an occupant of the vehicle is experiencing an increase in stress level. The program code portions further adapt an ADAS action of the vehicle to reduce an occupant stress level from a first level to a second level lower than the first level.

In a yet another aspect of the present disclosure the method further includes reducing computational resource usage from a first level to a second level lower than the first level.

In a yet another aspect of the present disclosure the method further includes executing a machine-learning algorithm using Shapley Additive Explanations (SHAP), to select the subset of the sensor data corresponding to the subset of the one or more physiological sensors.

In a yet another aspect of the present disclosure the method further includes maintaining accuracy of stress level predictions while using the subset of the sensor data from the subset of the one or more physiological sensors.

In a yet another aspect of the present disclosure the method further includes utilizing a machine-learning algorithm comprising long short-term memory (LSTM) to predict the stress level of the occupant of the vehicle.

In a yet another aspect of the present disclosure the method actively, recursively, and continuously modulates the ADAS action to reduce the occupant stress level from the first level to the second level upon detecting an increase in occupant stress level in response to an ADAS action of the vehicle.

In a yet another aspect of the present disclosure the method further collects sensor data from one or more of: electroencephalographs (EEGs), functional near-infrared spectroscopy (fNIRS) sensors, galvanic skin response (GSR) sensors, pupil diameter sensors, heart rate sensors, eye movement sensors, thermal camera, web camera, and photoplethysmography (PPG) sensors.

In a yet another aspect of the present disclosure the method collects sensor data from the GSR sensor detecting changes in galvanic skin response, the pupil diameter sensor detecting changes in diameter of the occupant's pupil, and eye movement sensors detecting changes in eye position and rate of movement in X and Y directions.

In a yet another aspect of the present disclosure the method actively informs the occupant, via a human-machine interface, of planned adaptations to the ADAS actions of the vehicle.

In a yet another aspect of the present disclosure a system for dynamically adjusting interactions between an advanced driver assistance system (ADAS) equipped vehicle and occupants of the vehicle includes one or more physiological sensors disposed on the vehicle. The one or more physiological sensors include: electroencephalographs (EEGs), functional near-infrared spectroscopy (fNIRS) sensors, galvanic skin response (GSR) sensors, pupil diameter sensors, heart rate sensors, eye movement sensors, and photoplethysmography (PPG) sensors. The system further includes one or more control modules having a processor, a memory, and input/output (I/O) ports, the I/O ports in communication with the one or more physiological sensors. The control modules execute program code portions stored in the memory. The program code portions include a first program code portion that collects sensor data from the one or more physiological sensors, and a second program code portion that analyzes the sensor data and selects a subset of the sensor data corresponding to a subset of the one or more physiological sensors such that computational resource usage is decreased from a first resource consumption level to a second resource consumption level less than the first resource consumption level. The system selects the subset of sensor data using a machine-learning algorithm using Shapley Additive Explanations (SHAP) to select the subset of the sensor data corresponding to the subset of the one or more physiological sensors, and a machine-learning algorithm using long short-term memory (LSTM) to predict the stress level of the occupant of the vehicle. The subset of physiological sensors includes: the GSR sensor detecting changes in galvanic skin response, the pupil diameter sensor detecting changes in diameter of the occupant's pupil, and eye movement sensors detecting changes in eye position and rate of movement in X and Y directions. The system program code portions further include a third program code portion that predicts, based on the subset of the sensor data, that an occupant of the vehicle is experiencing an increase in stress level. The program code portions further include a fourth program code portion that upon detecting an increase in occupant stress level in response to an ADAS action of the vehicle, actively, recursively, and continuously modulates the ADAS action to reduce an occupant stress level from a first level to a second level lower than the first level. The program code portions further include a fifth program code portion that actively informs the occupant, via a human-machine interface, of planned adaptations to the ADAS actions of the vehicle.

In a yet another aspect of the present disclosure the third program code portion maintains accuracy of stress level predictions while using subset of the sensor data from the subset of the one or more physiological sensors.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a schematic diagram of a system for emotional state assessment with dynamic use of physiological sensor responses to enhance situational awareness for human machine interaction according to an aspect of the present disclosure;

FIG. 2A is a block diagram depicting a calibration algorithm for the system for emotional state assessment with dynamic use of physiological sensor responses to enhance situational awareness for human machine interaction of FIG. 1 according to an aspect of the present disclosure;

FIG. 2B is a block diagram depicting an exemplary use case of the system for emotional state assessment with dynamic use of physiological sensor responses to enhance situational awareness for human machine interaction of FIG. 1 according to an aspect of the present disclosure;

FIG. 3 is a flowchart depicting a method for emotional state assessment with dynamic use of physiological sensor responses to enhance situational awareness for human machine interaction according to an aspect of the present disclosure; and

FIG. 4 is a chart depicting exemplary physiological sensor outputs of the system and method for emotional state assessment with dynamic use of physiological sensor responses to enhance situational awareness for human machine interaction according to an aspect of the present disclosure.

### DETAILED DESCRIPTION

The following description is merely exemplary in nature and is not intended to limit the present disclosure, application, or uses.

In the claims and specification, certain elements are designated as “first”, “second”, “third”, “fourth”, “fifth”, “sixth”, and “seventh”, etc. These are arbitrary designations intended to be consistent only in the section in which they appear, i.e. the specification or the claims or the summary, and are not necessarily consistent between the specification, the claims, and the summary. In that sense they are not intended to limit the elements in any way and a “second” element labeled as such in the claim may or may not refer to a “second” element labeled as such in the specification. Instead, the elements are distinguishable by their disposition, description, connections, and function.

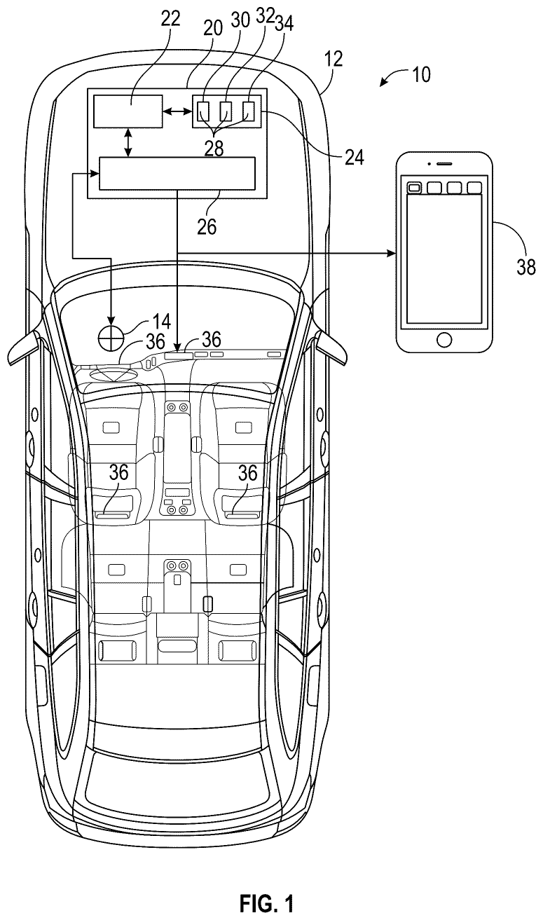

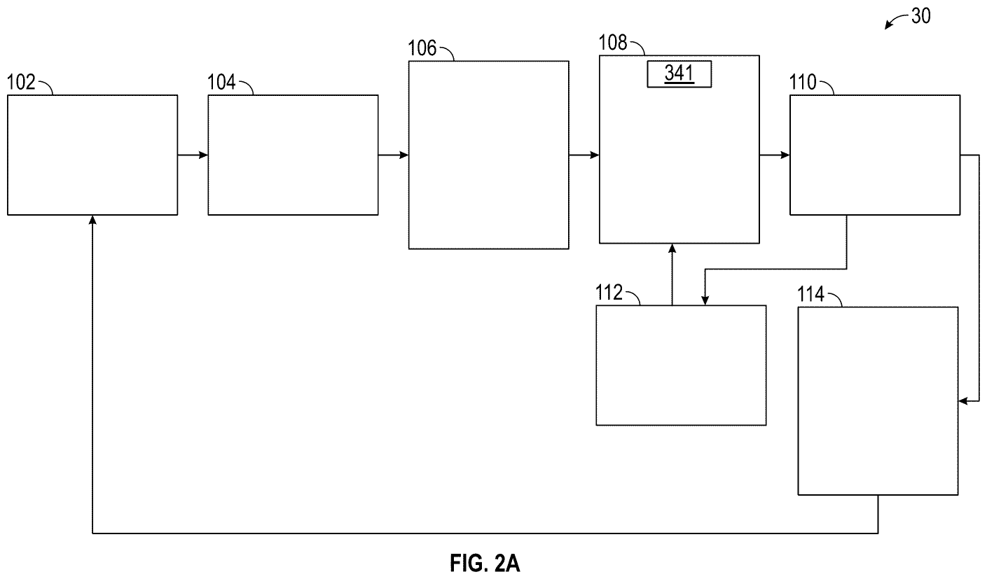

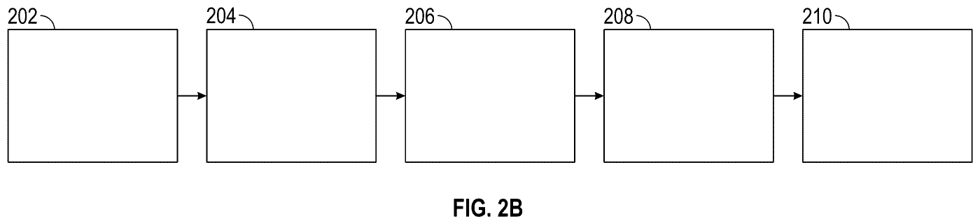

Referring now to FIGS. 1 and 2, a system 10 for assessing emotional states of vehicle 12 users is shown. The system 10 utilizes a variety of onboard physiological sensors 14 disposed on the vehicle 10 to gather sensor data regarding an emotional state of a vehicle 12 user, and processes the sensor data within one or more control modules 20 to determine the emotional state of the vehicle 12 users.

The one or more control modules 20 are non-generalized, electronic control devices having a preprogrammed digital computer or processor 22, non-transitory computer readable medium or memory 24 used to store data such as control logic, software applications, instructions, computer code, data, lookup tables, etc., and a transceiver or input/output (I/O) ports 26. Memory 24 includes any type of medium capable of being accessed by a computer, such as read only memory (ROM), random access memory (RAM), a hard disk drive, a compact disc (CD), a digital video disc (DVD), or any other type of memory. A “non-transitory” computer readable medium or memory 24 excludes wired, wireless, optical, or other communication links that transport transitory electrical or other signals. A non-transitory computer memory 24 includes media where data may be permanently stored and media where data can be stored and later overwritten, such as a rewritable optical disc or an erasable memory device. Computer code includes any type of program code, including source code, object code, and executable code. The processor 22 is configured to execute the code or instructions. The control modules 20 may also be dedicated Wi-Fi controllers, engine control modules, transmission control modules, body control modules, infotainment control modules, or the like. The I/O ports 26 are configured to communicate wirelessly or through wired means with known means, including Wi-Fi protocols under IEEE 802.11x.

The control modules 20 may further include one or more applications 28. An application 28 is a software program configured to perform a specific function or set of functions. The application 28 may include one or more computer programs, algorithms, software components, sets of instructions, procedures, functions, objects, classes, instances, related data, or a portion thereof adapted for implementation in a suitable computer readable program code. The applications 28 may be stored within the memory 24 or in additional or separate memory 24.

Turning now specifically to FIGS. 2A and 2B, and with continuing reference to FIG. 1, on several aspects, one or more of the applications 28 includes a calibration algorithm 30. The calibration algorithm 30 may be stored in a memory 24 within a control module 20 of the vehicle 12, in a remotely-located control module 20, or both. Physiological sensors 14 used with the calibration algorithm 30 may include physiological sensors 14 used to collect electroencephalographs (EEGs), functional near-infrared spectroscopy (f N IRS) data regarding oxygen usage via a forehead scan, galvanic skin response (GSR), pupil diameter, heart rate, eye movement broken into eye gaze in the X and Y directions, thermal data, and photoplethysmography (PPG) data, or the like.

The calibration algorithm 30 collects sensor data from the physiological sensors 14 at block 102 during a period of time in which a vehicle 12 user experiences one or more stress-inducing events during a calibration simulation. In several aspects, the stress-inducing event may be a simulated situation in which a pedestrian walks in front of the vehicle 12 as the vehicle 12 is driven towards the pedestrian, or where the vehicle 12 is being driven through heavy precipitation at speed, or the like. The sensor data from the physiological sensors 14 is then filtered and preprocessed and normalized at blocks 104 and 106. More specifically, at block 106, additional features such as EEG frequency bands, wavelet-filtered fNIRS, or the like are used to further filter and preprocess the sensor data from the physiological sensors 14. At block 108, the outputs from block 106 is used to train a machine learning model 34 that predicts when a vehicle 12 user will experience stress. A machine learning output explanation method is then used at block 110 to detect which sensor data signals, and which time intervals during the stress-inducing event were most important in the machine learning model's 34 predictions at block 108. At block 112, after using the machine learning output explanation method, only those physiological sensor 14 signals and time intervals identified as most important are retained and used to retrain the calibration algorithm 30. The calibration algorithm 30 is then retrained recursively with fewer features at block 108, and physiological sensor 14 data importance is calculated once more at block 112. It should be appreciated that each vehicle 12 occupant or operator has a different physiology than other users or operators. Since each occupant or operator may therefore provide differing physiological signals via the physiological sensors 14, the system 10 adapts or calibrates to each user's or operator's individual physiological stress response characteristics. Accordingly, each occupant or operator may produce differing signals or signal quality, and the system 10 actively and continuously calibrates to the signals and signal quality provided by each occupant or operator of the vehicle 12 while the system 10 is operational. Likewise, the system 10 may provide a personalized emotional state prediction according to an individual user's or operator's physiological signals and signal quality.

Once the calibration algorithm 30 is completed, the resulting machine learning model 34 is used online and in real-time at during operation of the vehicle 12 at block 114 to predict emotional states of a vehicle 12 user using only the signals that were identified as most important during calibration. Data from online use of the machine learning model 34 at block 114 is returned to the calibration algorithm 30 at block 102 and incorporated so that the vehicle 12 continues to learn while the vehicle 12 is in operation.

Turning now to FIG. 2B, and with continuing reference to FIGS. 1-2A, predictions from the machine learning model 34 are used to modulate vehicle 12 behavior and communicate with vehicle 12 users while advanced driver assistance systems (ADAS) are in use. Examples of ADAS may include, but are not limited to: lane keep assistance systems, cruise control systems, traffic avoidance systems, obstacle avoidance systems, automated parking systems, and the like. In several aspects, upon activation and use of an ADAS within the vehicle 12, vehicle occupants may experience increased stress levels due to ADAS-generated vehicle 12 actions. For example, at block 202 the vehicle 12 may take an ADAS-based action that causes a stress response in the vehicle 12 users. At block 204, the physiological sensors 14 of the vehicle 12 detect the stress response as an increase in vehicle 12 user stress levels due to the vehicle's 12 driving behavior. Based on such a detected increase in vehicle 12 user stress, at block 206, the system 10 identifies relevant physiological sensors 14 that are most likely to provide accurate data regarding vehicle 12 user stress levels. At block 208, the system 10 utilizes the machine-learning model 34 to instruct the vehicle 12 to alter the ADAS-based action to reduce the user's stress level according to predetermined stress-correlated information. For example, when the ADAS-based action is a cruise-control action at highway speeds, and the user or occupant of the vehicle 12 is detectably stressed, the system 10 may use the machine-learning model 34 to instruct the vehicle 12 to reduce the speed at which the vehicle 12 is being driven. In several aspects, changes in occupant or user stress levels may be detected via eye gaze information, pupil diameter changes, galvanic skin response, heart rate, thermal data, or PPG data, or the like. At block 210, the system 10 indicates the planned stress-reducing action through an occupant-notification mechanism such as displaying the planned action on a human-machine interface (HMI) 36 of the vehicle 12, a third-party device such as a cellular device 38, tablet, or the like.

The system 10 may be trained using a machine-learning model such as Long Short-Term Memory LSTM as the basis for the predictive machine-learning model 34. The machine-learning model may further include Shapley Additive Explanations (SHAP) that generate heat maps that are used to selectively determine which of the various physiological sensors 14 are relevant to determining stress levels of the occupants of the vehicle 12. The LSTM operates on a time-series prediction, and can be used in an offline training fashion, or modified for online learning without departing from the scope or intent of the present disclosure. SHAP utilizes a game theoretic approach to explain the output of the machine learning models 34.

In a test example used to train the machine learning model 34, physiological data is collected from several test subject vehicle 12 occupants. Each test subject is exposed to a video during which the subject is driving and another vehicle 12 approaches rapidly from behind and subsequently cuts in front of the test subject's vehicle 12. 8-channel EEG data, fNIRS, galvanic skin response, pupil diameter, PPG, thermal data, heart rate, and eye gaze physiological data are collected during the test example. The physiological data is preprocessed by dropping outliers, resampling the data streams to 128 Hz, applying a 1-50 Hz bandpass filter to the EEG channels, averaging left and right pupil diameters, and selecting the least noisy fNIRS channels. The data is subsequently normalized for the LSTM, sklearn StandardScalar is used individually for each test subject.

An LSTM is then used to predict whether not the next time sample includes the vehicle approach and cut-off from the test example, given the previous 64 samples, or half second of data. Since there are only 16 data streams from the various physiological sensors 14, each data point consists of a 64×16 matrix. Overlapping samples are used to preserve data.

After training the LSTM, SHAP determines which parts of the 64×16 feature matrix are most important for the LSTM predictions. From SHAP, the LSTM only uses the last half of time samples, and primarily utilizes pupil response, gaze in the X-direction, and channel 8 of the EEG in its predictions. In several aspects, channel 8 of the EEG detects activity at outer corners of a test subject's eyes. It should be appreciated that other physiological sensors 14 may be useful in the calculations above and those to follow. However, it should be further appreciated that not all measurable stress responses are as useful as others because response times between stress-inducing event and stress-induced measurable response may vary substantially depending on what is being measured. That is, human physiology causes some stress-response time lag. A test subject or an operator/user of the vehicle 12 may have near-instantaneous pupillary response or galvanic skin response (GSR) but heart rate may be substantially delayed by comparison. Likewise, a person's blood pressure and/or heart rate may increase in response to a stress-inducing event, but the time in which a measurable change in blood pressure and/or heart rate occurs is significantly greater than the amount of time a change in muscle movement at the outer corner of the person's eye may take. Accordingly, physiological sensors 14 that can detect rapid changes in response to stress-inducing events may be pre-selected for the calculations above and below in order to refine and simplify the system 10.

SHAP provides means to reduce a time window used to perform predictions to roughly 32 samples, rather than the full 64, as well as reducing a quantity of data signal streams to 4 from the full 16 without any significant loss in prediction accuracy. Accordingly, the input feature matrix may be reduced using SHAP from 64×16 to 32×4, thereby substantially reducing the number physiological sensors 14 needed, and substantially improving processing speed, reducing computational time and reducing the computational resources necessary from a first level to a second level less than the first level, while maintaining or improving the accuracy of the vehicle 12 operator emotional state predictions disclosed herein.

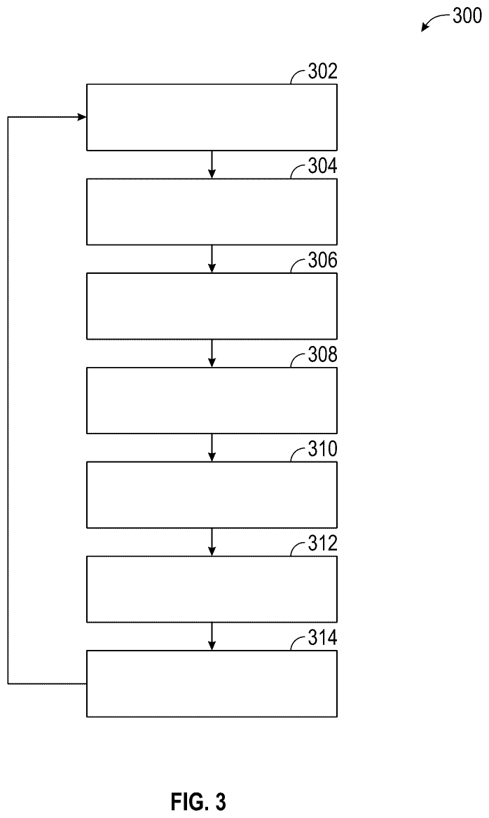

Turning now to FIG. 3 a flowchart depicting a method 300 for how the emotional state estimation is carried out by the system 10 of the present disclosure is shown. The method 300 begins at block 302 where the system 10 receives input from the various physiological sensors 14 equipped to the vehicle 12. In several aspects, the input is a 128×16 matrix including all data collected by each of 16 exemplary physiological sensors 14 at 128 Hz. At block 304, the system 10 uses the LSTM to hold the input data still in a 128×16 matrix. From block 304, the system 10 performs a batch normalization at block 306 to further refine the input data. At block 308, the system 10 again uses the LSTM to hold the batch normalized input data as 16 distinct data points. At block 310, the system applies a further batch normalization process to the data from block 308, and the output of block 310 remains 16 data points. At block 312, the system 10 densifies the data from block 310 and reduces the 16 data points down to a single data point output shown at block 314. Moreover, at block 314, the system 10 alters an ADAS vehicle 12 function to reduce stress on the user or occupant of the vehicle and indicates the planned stress-reducing action through an occupant-notification mechanism such as displaying the planned action on a human-machine interface (HMI) 36 of the vehicle 12, a third-party device such as a cellular device 38, tablet, or the like.

From block 314, the method 300 proceeds back to block 302 where the method 300 runs continuously, iteratively, and recursively to further refine emotional state estimations as well as to continuously improve the ADAS functions of the vehicle 12. Furthermore, through the method 200, the quantity of physiological sensors 14 utilized may be reduced without any loss in testing accuracy. That is, because the system 10 and method 300 of the present disclosure automatically identifies physiological sensors 14 and sensor data and continuously, iteratively, and recursively retrains machine learning model 34, the system 10 and method 300 of the present disclosure constantly improves the accuracy of its predictions.

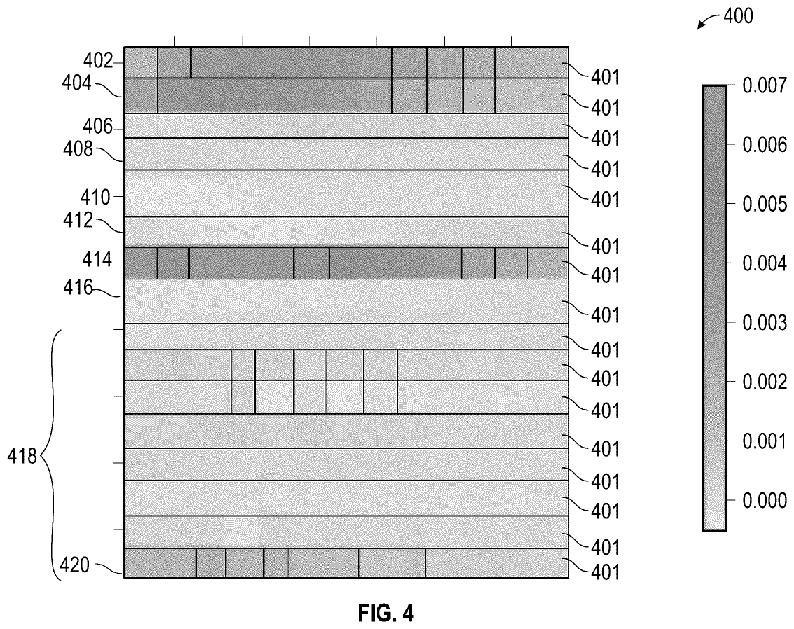

Turning now to FIG. 4 and with continuing reference to FIGS. 1-3, a chart 400 of output data from exemplary physiological sensors 14 used by the system 10 is shown in additional detail. More specifically, the chart 400 depicts data traces 401 from each of the exemplary physiological sensors 14 during a test scenario in which a test subject experiences a stress-inducing event such as that described hereinabove. In the chart 400, the darker in shading the traces 401 are, the greater the test subject's measurable stress-response. In several aspects, the physiological sensors 14 producing the data in the chart 400 are: pupillary response sensor 402, an eye position sensor 404, 406 providing gaze information in the X and Y directions, a pulse oximeter 408, a blood pressure sensor 410, a PPG sensor 412, a galvanic skin response sensor 414, a heart rate monitor 416, and an 8-channel EEG sensor 418 in which channel 8 is of particular note. Additional sensors may include thermal cameras (not specifically shown), web cameras (not specifically shown), and the like. As can be seen in the chart 400, pupil response 402, gaze in the X direction 404, galvanic skin response 414, and channel eight 420 of the EEG sensor 418 depict the darkest traces 401 and are therefore indicative of measurable stress increases during a stress-inducing event while the lighter traces 401 indicate that stress signals caused by the stress-inducing event may not be measurable by the sensors 14 producing those traces 401.

A system and method for emotional state assessment with dynamic use of physiological sensor 14 responses to enhance situational awareness for human-machine interaction according to the present disclosure offers several advantages. These include the ability to use low-cost hardware operating dynamically and in real-time with one or more physiological sensors 14 to assess the emotional state of vehicle 12 occupants to enhance situational awareness for human machine interaction while ADAS functions are being executed. The system and method of the present disclosure also improve ADAS functions by increasing safety and occupant comfort while decreasing computational resource usage without increasing manufacturing, hardware, or other such costs.

The description of the present disclosure is merely exemplary in nature and variations that do not depart from the gist of the present disclosure are intended to be within the scope of the present disclosure. Such variations are not to be regarded as a departure from the spirit and scope of the present disclosure.

What is claimed is:

1. A system for dynamically adjusting interactions between an advanced driver assistance system (ADAS) equipped vehicle and occupants of the vehicle, the system comprising:
one or more physiological sensors disposed on the vehicle;
one or more control modules having a processor, a memory, and input/output (I/O) ports, the I/O ports in communication with the one or more physiological sensors; the control modules executing program code portions stored in the memory, the program code portions comprising:
a first program code portion that collects sensor data from the one or more physiological sensors;
a second program code portion including a machine learning algorithm that analyzes the sensor data and selects a subset of the sensor data corresponding to a subset of the one or more physiological sensors;
a third program code portion that predicts, based on the subset of the sensor data, that an occupant of the vehicle is experiencing an increase in stress level; and
a fourth program code portion that adapts an ADAS action of the vehicle to reduce an occupant stress level from a first level to a second level lower than the first level.

2. The system of claim 1 wherein the second program code portion further selects the subset of the sensor data corresponding to the subset of the one or more physiological sensors such that computational resource usage is maintained or decreased from a first computational resource usage level to a second computational resource usage level less than the first computational resource usage level.

3. The system of claim 1 wherein the second program code portion comprises Shapley Additive Explanations (SHAP) to select the subset of sensor data corresponding to the subset of the one or more physiological sensors.

4. The system of claim 1 wherein the third program code portion maintains accuracy of stress level predictions while using subset of the sensor data from the subset of the one or more physiological sensors.

5. The system of claim 1 wherein the second program code portion comprises long short-term memory (LSTM) to predict the stress level of the occupant of the vehicle.

6. The system of claim 1 wherein upon detecting an increase in occupant stress level in response to an ADAS action of the vehicle, the fourth program code portion actively, recursively, and continuously modulates the ADAS action to reduce the occupant stress level from the first level to the second level.

7. The system of claim 1 wherein the one or more physiological sensors comprise: electroencephalographs (EEGs), functional near-infrared spectroscopy (fNIRS) sensors, galvanic skin response (GSR) sensors, pupil diameter sensors, heart rate sensors, eye movement sensors, thermal camera, web camera, and photoplethysmography (PPG) sensors.

8. The system of claim 7 wherein the subset of the one or more physiological sensors comprises the GSR sensor detecting changes in galvanic skin response, the pupil diameter sensor detecting changes in diameter of the occupant's pupil, and eye movement sensors detecting changes in eye position and rate of movement in X and Y directions.

9. The system of claim 1 further comprising a fifth control logic that actively informs the occupant, via a human-machine interface, of planned adaptations to the ADAS actions of the vehicle.

10. A method for dynamically adjusting interactions between an advanced driver assistance system (ADAS) equipped vehicle and occupants of the vehicle, the method comprising:
collecting sensor data from one or more physiological sensors disposed on the vehicle;
utilizing one or more control modules having a processor, a memory, and input/output (I/O) ports, the I/O ports in communication with the one or more physiological sensors; the control modules executing program code portions stored in the memory, the program code portions:
analyzing the sensor data and selecting a subset of the sensor data corresponding to a subset of the one or more physiological sensors;
predicting, based on the subset of the sensor data, that an occupant of the vehicle is experiencing an increase in stress level; and
adapting an ADAS action of the vehicle to reduce an occupant stress level from a first level to a second level lower than the first level.

11. The method of claim 10 wherein selecting the subset of the sensor data further comprises: reducing computational resource usage from a first level to a second level lower than the first level.

12. The method of claim 10 wherein analyzing the sensor data and selecting a subset of the sensor data further comprises:
executing a machine-learning algorithm using Shapley Additive Explanations (SHAP), to select the subset of the sensor data corresponding to the subset of the one or more physiological sensors.

13. The method of claim 10 further comprising:
maintaining accuracy of stress level predictions while using the subset of the sensor data from the subset of the one or more physiological sensors.

14. The method of claim 10 wherein analyzing the sensor data and selecting a subset of the sensor data further comprises
utilizing a machine-learning algorithm comprising long short-term memory (LSTM) to predict the stress level of the occupant of the vehicle.

15. The method of claim 10 wherein upon detecting an increase in occupant stress level in response to an ADAS action of the vehicle, actively, recursively, and continuously modulating the ADAS action to reduce the occupant stress level from the first level to the second level.

16. The method of claim 10 further comprising:
collecting sensor data from one or more of: electroencephalographs (EEGs), functional near-infrared spectroscopy (fNIRS) sensors, galvanic skin response (GSR) sensors, pupil diameter sensors, heart rate sensors, eye movement sensors, thermal camera, web camera, and photoplethysmography (PPG) sensors.

17. The method of claim 16 wherein selecting a subset of the sensor data corresponding to a subset of the one or more physiological sensors further comprises: collecting sensor data from the GSR sensor detecting changes in galvanic skin response, the pupil diameter sensor detecting changes in diameter of the occupant's pupil, and eye movement sensors detecting changes in eye position and rate of movement in X and Y directions.

18. The method of claim 10 further comprising: actively informing the occupant, via a human-machine interface, of planned adaptations to the ADAS actions of the vehicle.

19. A system for dynamically adjusting interactions between an advanced driver assistance system (ADAS) equipped vehicle and occupants of the vehicle, the system comprising:
one or more physiological sensors disposed on the vehicle, the one or more physiological sensors including: electroencephalographs (EEGs), functional near-infrared spectroscopy (fNIRS) sensors, galvanic skin response (GSR) sensors, pupil diameter sensors, heart rate sensors, eye movement sensors, and photoplethysmography (PPG) sensors;
one or more control modules having a processor, a memory, and input/output (I/O) ports, the I/O ports in communication with the one or more physiological sensors; the control modules executing program code portions stored in the memory, the program code portions comprising:
a first program code portion that collects sensor data from the one or more physiological sensors;
a second program code portion that analyzes the sensor data and selects a subset of the sensor data corresponding to a subset of the one or more physiological sensors such that computational resource usage is decreased from a first resource consumption level to a second resource consumption level less than the first resource consumption level using a machine-learning algorithm using Shapley Additive Explanations (SHAP) to select the subset of the sensor data corresponding to the subset of the one or more physiological sensors, and a machine-learning algorithm using long short-term memory (LSTM) to predict the stress level of the occupant of the vehicle, wherein the subset of physiological sensors comprises: the GSR sensor detecting changes in galvanic skin response, the pupil diameter sensor detecting changes in diameter of the occupant's pupil, and eye movement sensors detecting changes in eye position and rate of movement in X and Y directions;
a third program code portion that predicts, based on the subset of the sensor data, that an occupant of the vehicle is experiencing an increase in stress level; and
a fourth program code portion that upon detecting an increase in occupant stress level in response to an ADAS action of the vehicle, the fourth program code portion actively, recursively, and continuously modulates the ADAS action to reduce an occupant stress level from a first level to a second level lower than the first level; and
a fifth program code portion that actively informs the occupant, via a human-machine interface, of planned adaptations to the ADAS actions of the vehicle.

20. The system of claim 19 wherein the third program code portion maintains accuracy of stress level predictions while using subset of the sensor data from the subset of the one or more physiological sensors.
